(19) United States
(12) Patent Application Publication
(10) Pub. No.: US 2007/0282213 A1
(43) Pub. Date: Dec. 6, 2007

(54) Apparatus and computer program for assessing a patient's extravascular lung water volume

(75) Inventors: Frederic Michard, Bievres (FR); Ulrich Pfeiffer, Muenchen (DE); Reinhold Knoll, Muenchen (DE)

Correspondence Address: DAVIDSON, DAVIDSON & KAPPEL, LLC, 485 SEVENTH AVENUE, 14TH FLOOR, NEW YORK, NY, 10018, US

(73) Assignee: UP Management GmbH & Co Med-Systems KG, Muenchen (DE)

(21) Appl. No.: 11/799,645

(22) Filed: May 2, 2007

(30) Foreign Application Priority Data
May 5, 2006 (DE) 10 2006 021 034.4

(51) Int. Cl. A61B 5/08

(52) U.S. Cl. 600/529

(57) ABSTRACT

A method and apparatus for assessing extravascular lung water volume (EVLW) of a patient after lung resection is adapted to provide a transpulmonary thermodilution curve, is capable to derive the patient's intrathoracic thermal volume (ITTV) and approximated intrathoracic blood volume (ITBVₐₚₚᵣₒₓ) from the transpulmonary thermodilution curve, and is capable to determine the extravascular lung water volume (EVLW) by correcting by the degree of lung resection (c), wherein making use of the equation EVLW=f(ITTV, ITBVₐₚₚᵣₒₓ, c), or EVLW=f(ITTV, GEDV, c). Further, a computer program for assessing extravascular lung water volume (EVLW) of a patient after lung resection, has instructions adapted to carry out the steps of generating a transpulmonary thermodilution curve on basis of provided measurement data collected at the patient, deriving the patient's intrathoracic thermal volume (ITTV) and approximated intrathoracic blood volume (ITBVₐₚₚᵣₒₓ) from the transpulmonary thermodilution curve, and determining the extravascular lung water volume (EVLW) by correcting by the degree of lung resection (c), wherein making use of the equation EVLW=f(ITTV, ITBVₐₚₚᵣₒₓ, c) or EVLW=f(ITTV, GEDV, c), when run on a computer.

This claims the benefit of German Patent Application No. DE 10 2006 021 034.4, filed on May 5, 2006 and hereby incorporated by reference herein.

The invention relates to an apparatus and a computer program for observing a patient's life-threatening condition caused by pulmonary edema by assessing the patient's extravascular lung water volume.

### BACKGROUND

It is generally known that a patient suffering from pulmonary edema is in a life threatening condition. Such a pulmonary edema may result from an increase in hydrostatic pulmonary capillary pressure (cardiogenic pulmonary edema) and/or from an increase in pulmonary capillary permeability (acute lung injury, Acute Respiratory Distress Syndrome).

In the critical-care diagnostics and treatment of such a patient it is useful to use the patient's extravascular lung water volume EVLW as an important characteristic for monitoring the patient's state of health to guide therapy and to improve the outcome thereof.

In general, a normal value of extravascular lung water volume EVLW of a healthy person ranges between 3 and 7 ml/kg. However, a patient suffering from an increase in hydrostatic pulmonary capillary pressure (cardiogenic pulmonary edema) and/or from an increase in pulmonary capillary permeability (acute lung injury, Acute Respiratory Distress Syndrome) leading to a pulmonary edema may has a value of extravascular lung water volume EVLW reaching 25 to 30 ml/kg.

For accurate determination of extravascular lung water volume EVLW of an animal the usage of an ex-vivo post-mortem gravimetric method is known. By nature this method can only be employed once and, of course, is not applicable in humans for repeated monitoring of EVLW.

According to findings in literature, for purposes of monitoring the extravascular lung water volume EVLW can repeatedly be derived from the difference between the intrathoracic thermal volume ITTV and the intrathoracic blood volume ITBV of the patient, i.e.

EVLW=ITTV−ITBV.

The intrathoracic thermal volume ITTV and the intrathoracic blood volume ITBV can be determined using a transpulmonary thermodilution analysis method, as it is proposed in U.S. Pat. No. 5,526,817. A bolus of an indicator defined by a predetermined quantity of the indicator is injected into the patent's vena cava superior, and the indicator concentration response is measured at a downstream location of the patient's systemic circulation, e.g. at the patient's femoral artery. Based on the indicator concentration response measurement versus time the transpulmonary thermodilution curve is generated. The intrathoracic thermal volume ITTV and the Global End Diastolic Volume GEDV are derived from the mathematical analysis of the transpulmonary thermodilution curve according to methods known from prior art. It has been established that the intrathoracic blood volume ITBV and the global end-diastolic volume GEDV are closely related and that the intrathoracic blood volume ITBV can be approximated from the global end-diastolic volume GEDV by using the equation

ITBVₐₚₚᵣₒₓ=a GEDV+b.

The extravascular lung water volume EVLW can therefore be derived from the difference between the intrathoracic thermal volume ITTV and the approximated intrathoracic blood volume ITBV of the patient, i.e.

EVLW=ITTV−ITBVₐₚₚᵣₒₓ.

It is desirable that above mentioned method for assessing the extravascular lung water volume EVLW of a patient is accurate and hence reliable for monitoring the patient's state of health to guide therapy and to improve the outcome thereof when applying such method to a patient suffering from pulmonary edema and having parts of his/her lung resected. However, the circumstance of lung resection affects the accuracy of results obtained by a transpulmonary thermodilution method. Therefore, when using a thermodilution method for determining a value of the patient's extravascular lung water volume EVLW for diagnostics and therapy, this value is inaccurate and hence only less significant in the clinical-care point of view.

### SUMMARY OF THE INVENTION

It is an object of the invention to provide an apparatus and a computer program for observing a patient's life-threatening condition caused by pulmonary edema by assessing the patient's extravascular lung water volume in simple, but nevertheless exact and reliable way, although the patient has a resected lung.

According to the invention, this object is achieved by an apparatus for assessing extravascular lung water volume EVLW of a patient after lung resection, adapted to provide a transpulmonary thermodilution curve, capable to derive the patient's intrathoracic thermal volume ITTV and approximated intrathoracic blood volume ITBVₐₚₚᵣₒₓ from the transpulmonary thermodilution curve, and capable to determine the extravascular lung water volume EVLW by correcting said approximated intrathoracic blood volume ITBVₐₚₚᵣₒₓ by the degree of lung resection c, wherein making use of the equation

EVLW=f(ITTV, ITBVₐₚₚᵣₒₓ, c), or

EVLW=f(ITTV, GEDV, c).

Further, according to the invention, this object is achieved by a computer program for assessing extravascular lung water volume EVLW of a patient after lung resection. The computer program has instructions adapted to carry out the steps of generating a transpulmonary thermodilution curve on basis of provided measurement data collected at the patient, deriving the patient's intrathoracic thermal volume ITTV and approximated intrathoracic blood volume ITBVₐₚₚᵣₒₓ from the transpulmonary thermodilution curve, and determining the extravascular lung water volume EVLW by correcting said approximated intrathoracic blood volume ITBVₐₚₚᵣₒₓ by the degree of lung resection c, wherein making use of the equation

EVLW=f(ITTV, ITBVₐₚₚᵣₒₓ, c), or

EVLW=f(ITTV, GEDV, c),

when run on a computer.

Due to the fact that according to the invention the extravascular lung water volume EVLW is corrected on basis of the degree of lung resection c of the patient, the value of the extravascular lung water volume EVLW assessed by the inventive apparatus and computer program is accurate and reliable, although the value of the extravascular lung water volume EVLW is determined by making use of the easily feasible bedside method of thermodilution.

Preferably, the apparatus is adapted to estimate the degree of lung resection c by making use of an empirical average value of the reduction of pulmonary perfusion of the resected lung compared to the pre-operative lung of the patient. Further, according to a preferred embodiment of the invention, the computer program has instructions being adapted to carry out the step of this estimate.

As an alternative, it is preferred that the apparatus and the computer program make use of, in case of a resected right lung, estimating the degree of lung resection c as being 55%, or, in case of a resected left lung, estimating the degree of lung resection c as being 45%, and/or, in case of resected lobes, estimating the degree of lung resection c as being the sum of normal perfusion rates of the resected lobes.

Normal perfusion rate right lung:

Right superior lobe 18%

Right middle lobe 12%

Right inferior lobe 25%

Normal perfusion rate left lung:

Left superior lobe 25%

Left inferior lobe 20%

As a further alternative, it is preferred that the apparatus is adapted to estimate the degree of lung resection c by comparing the pulmonary perfusion before and after the lung resection, wherein the pulmonary perfusion values are provided by making use of pre-operative perfusion lung scans and dividing the density of areas which will be resected by the density of the whole unresected lung. It is also a preferred alternative embodiment of the invention that the computer program has instructions adapted to carry out this estimate.

Preferably, the apparatus is adapted to make use of the equation

EVLW=ITTV−ITBV|_{corrected},

wherein ITBV|_{corrected} is the value of the approximated intrathoracic blood volume ITBVₐₚₚᵣₒₓ corrected by the degree of lung resection c. It is also preferred that the computer program has instructions adapted to carry out the step of making use of this equation.

It is preferred that the apparatus and the instructions of the computer program are adapted to make use of equating the degree of lung resection c with the degree of reduction of pulmonary blood volume PBV caused by the lung resection.

Further, the apparatus is adapted to derive the global end-diastolic volume GEDV of the patient from the transpulmonary thermodilution curve and is adapted to make use of the equation

ITBV|_{corrected}=GEDV+PBV|_{corrected},

wherein PBV|_{corrected} is the value of the pulmonary blood volume PBV corrected by the degree of lung resection c.

Preferably the computer program has instructions adapted to carry out the steps of deriving the global end-diastolic volume GEDV from the transpulmonary thermodilution curve and making use of the above equation.

Further, according to a preferred embodiment of the apparatus and the computer program, both are adapted to make use of the equation

PBV|_{corrected}=((a−1)GEDV+b)(100%−c[%]),

wherein a and b are parameters in particular set to be

a=1.25 and b=0.0.

### BRIEF DESCRIPTION OF THE DRAWING

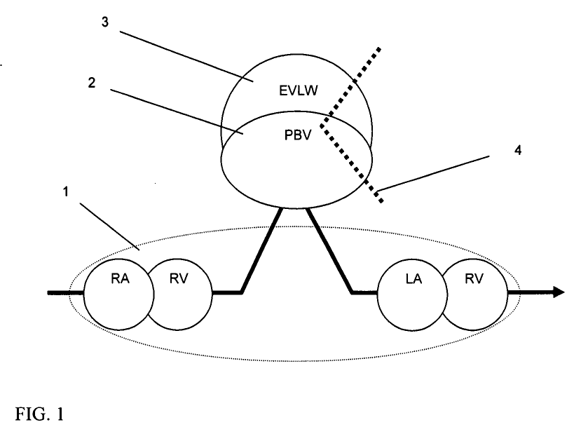

In the following the invention is explained on the basis of a preferred embodiment with reference to the drawing. In the drawing shows FIG. 1 a model of a pulmonary circulation.

### DETAILED DESCRIPTION

FIG. 1 shows a model of a pulmonary circulation comprising a heart and a lung. The right atrium RA of the heart, the right ventricle RV of the heart, the left atrium LA of the heart and the left ventricle LV of the heart define the sum of blood volumes outside the lung. This sum of blood volumes outside the lung defines the global end-diastolic volume GEDV designated with 1.

Further, the model of the pulmonary circulation comprises the pulmonary blood volume PBV designated with 2, and the extravascular lung water EVLW designated with 3.

To assess the extravascular lung water volume EVLW, a transpulmonary thermodilution method is used. A device as disclosed in U.S. Pat. No. 5,526,817, hereby incorporated by reference herein, can be used to perform the transpulmonary thermodilution method. The method is based on the physiologic relationship between the global end-diastolic volume GEDV and the intrathoracic blood volume ITBV. The global end-diastolic volume GEDV is derived from the mathematical analysis of the transpulmonary thermodilution curve.

It has been established that the intrathoracic blood volume ITBV and the global end-diastolic volume GEDV are closely related and that the intrathoracic blood volume ITBV can be approximated from the global end-diastolic volume GEDV by using the equation

ITBVₐₚₚᵣₒₓ=a GEDV+b,

wherein a and b are parameters, for example set to be a=1.25 and b=0.0.

The intrathoracic blood volume ITBV can be approximated from the global end-diastolic volume GEDV measurements. Therefore, the extravascular lung water volume EVLW can be estimated as the difference between the intrathoracic thermal volume ITTV and the approximated intrathoracic blood volume ITBVₐₚₚᵣₒₓ.

However, when a patient undergoes lung resection, the accuracy of this approximation from transpulmonary thermodilution is affected by the lung resection.

In FIG. 1 a lung resection cut which separates a part of the lung and of pulmonary blood volume PBV is shown and designated with 4.

The estimation of the extravascular lung water volume EVLW by the thermodilution method is based on the approximation of the intrathoracic blood volume ITBV from the global end-diastolic volume GEDV by using the equation

ITBVₐₚₚᵣₒₓ=a GEDV+b.

Further, the approximated pulmonary blood volume PBVₐₚₚᵣₒₓ is defined by the difference between the approximated intrathoracic blood volume ITBVₐₚₚᵣₒₓ and the global end-diastolic volume GEDV, i.e.

PBVₐₚₚᵣₒₓ=ITBVₐₚₚᵣₒₓ−GEDV.

When taking above equations into account, the pulmonary blood volume PBV can be expressed by

PBVₐₚₚᵣₒₓ=a GEDV+b−GEDV=b+(a−1)GEDV.

In case of lung resection, the pulmonary blood volume PBV is actually much lower than predicted by this equation. Therefore, in particular this decrease in pulmonary blood volume PBV affects the estimation of the intrathoracic blood volume ITBV and hence the estimation of the extravascular lung water volume EVLW.

Taking above equations into account, the intrathoracic blood volume ITBV is calculated as

ITBVₐₚₚᵣₒₓ=a GEDV+b=GEDV+PBVₐₚₚᵣₒₓ

1.=GEDV+b+(a−1)GEDV,

wherein [b+(a−1)GEDV] represents the approximated pulmonary blood volume PBVₐₚₚᵣₒₓ.

Most patients undergoing a lung resection are evaluated pre-operatively by a perfusion lung scan allowing a quantification of the actual perfusion of each lung (or lung lobe). From this distribution of pulmonary perfusion the relative perfusion of the removed part of the lung can be estimated. For example, if 55% of pulmonary perfusion was going to the right lung and 45% of the pulmonary perfusion to the left lung, it can be assumed that after resection of the right lung the pulmonary blood volume will be decreased by 55%.

This assumption can be done in a similar way for each of the three right lung lobes and each of the two left lung lobes. Therefore, the decrease in pulmonary blood volume PBV after the lobe resection can be also predicted. For example, if it is known from the preoperative perfusion lung scan that the inferior right lobe collects 25% of the pulmonary perfusion, it could be assumed that inferior right lobe surgical resection will reduce the pulmonary blood volume by 25%. This expected reduction of pulmonary blood volume PBV in relation to the pre-operative pulmonary blood volume PBV could be used as degree of resection c of total pulmonary perfusion caused by the resection representing the lung or lung lobe removed during the surgical procedure.

In case a perfusion lung scan is not available (e.g. use of transpulmonary thermodilution of a patient who underwent lung resection surgery several years ago), post-operative pulmonary blood volume PBV and the degree of resection c can be estimated from the normal anatomical distribution of pulmonary blood volume in human beings.

In order to improve the calculation of the intrathoracic blood volume ITBV, the degree of resection c has to be taken into consideration.

In general, a corrected intrathoracic blood volume ITBV|_{corrected} could be derived from the approximated intrathoracic blood volume ITBV|ₐₚₚᵣₒₓ and the degree of resection c, i.e.

ITBV|_{corrected}=f(ITBV|ₐₚₚᵣₒₓ, c).

When using the transpulmonary thermodilution method known from U.S. Pat. No. 5,526,817, the pulmonary blood volume PBV can be calculated as

PBV_{corrected}=(b+(a−1)GEDV)×(100%−c[%])

and the intrathoracic blood volume ITBV can be calculated as

ITBV|_{corrected}=GEDV+PBV_{corrected}=

1.=GEDV+(b+(a−1)GEDV)(100%−c[%]).

or as

ITBV|_{corrected}=ITBV|ₐₚₚᵣₒₓ−c[%](b+(a−1)GEDV).

For example, if a=1.25, b=0.0, and c=45% (e.g. surgical resection of a left lung representing 45% of the total pulmonary perfusion on a preoperative lung scan), the corrected intrathoracic blood volume ITBV_{corrected} is calculated as

ITBV|_{corrected}=1.1375GEDV

Therefore, modifying the equation to derive ITBV_{corrected} from GEDV according to the degree of resection c easily overcomes the current limitation of the transpulmonary thermodilution method in estimating the extravascular lung water volume EVLW after lung resection.

Taking above mentioned and described modelling into account, a process for assessing extravascular lung water volume EVLW of a patient after lung resection comprises the steps of:

Generating a transpulmonary thermodilution curve on basis of provided measurement data collected from the patient.

Deriving the patient's intrathoracic thermal volume ITTV and approximated intrathoracic blood volume ITBVₐₚₚᵣₒₓ from the transpulmonary thermodilution curve based on relations derived with unresected lungs.

Estimating the degree of lung resection c by making use of an empirical average value of the reduction of pulmonary perfusion of the resected lung compared to the pre-operative lung of the patient, or, in case of a resected right lung, estimating the degree of lung resection c as being 55%, or, in case of a resected left lung, estimating the degree of lung resection c as being 45%. Or estimating the degree of lung resection c by comparing the pulmonary perfusion before and after the lung resection, wherein the pulmonary perfusion values are provided by making use of pre-operative perfusion lung scans and dividing the density of areas which will be resected by the density of the whole unresected lung. Or in case of resected lobes, estimating the degree of lung resection c as being the sum of normal perfusion rates of the resected lobes.

Normal perfusion rate right lung:

Right superior lobe 18%

Right middle lobe 12%

Right inferior lobe 25%

Normal perfusion rate left lung:

Left superior lobe 25%

Left inferior lobe 20%

Determining the corrected intrathoracic blood volume ITBV_{corrected} by correcting for the degree of lung resection c, wherein making use of the equation

ITBV_{corrected}=f(ITBVₐₚₚᵣₒₓ, c).

Making use of the equation

EVLW=ITTV−ITBV|_{corrected},

wherein ITBV|_{corrected} is the value of the intrathoracic blood volume ITBV corrected by the degree of lung resection c.

Making use of equating the degree of lung resection c with the degree of reduction of pulmonary blood volume PBV caused by the lung resection.

Deriving the global end-diastolic volume GEDV of the patient from the transpulmonary thermodilution curve.

Making use of the equation

ITBV|_{corrected}=GEDV+PBV|_{corrected},

wherein PBV|_{corrected} is the value of the pulmonary blood volume PBV corrected by the degree of lung resection c.

Making use of the equation

PBV|_{corrected}=((a−1)GEDV+b)(100%−c%),

wherein a and b are parameters.

Setting the parameters a and b to be

a=1.25 and b=0.0.

The device as disclosed in U.S. Pat. No. 5,526,817 can be modified by programming a processor to calculate the EVLW, or a processor can be provided to receive inputs from such a device and this processor can calculate the EVLW.

What is claimed is:

1. An apparatus for assessing extravascular lung water volume (EVLW) of a patient after lung resection, comprising:
a device adapted to provide a transpulmonary thermodilution curve, capable of deriving the difference between the patient's intrathoracic thermal volume (ITTV) and approximated intrathoracic blood volume (ITBVₐₚₚᵣₒₓ) from the transpulmonary thermodilution curve, and capable of determining the extravascular lung water volume (EVLW) by correcting by a degree of lung resection (c), wherein making use of the equation
EVLW=f(ITTV, ITBVₐₚₚᵣₒₓ, c), or
EVLW=f(ITTV, GEDV, c),
GEDV being a global end-diastolic volume.

2. The apparatus according to claim 1, wherein the device is adapted to estimate the degree of lung resection (c) by making use of an empirical average value of the reduction of pulmonary perfusion of the resected lung compared to the pre-operative lung of the patient.

3. The apparatus according to claim 1, wherein in case of a resected right lung the device is adapted to estimate the degree of lung resection (c) as being 55%, or in case of a resected left lung the apparatus is adapted to estimate the degree of lung resection (c) as being 45%, and/or in case of a resected lobes, estimating the degree of lung resection c as being a sum of normal perfusion rates of the resected lobes.

4. The apparatus according to claim 1, wherein the device is adapted to estimate the degree of lung resection (c) by comparing the pulmonary perfusion before and after the lung resection, wherein the pulmonary perfusion values are provided by making use of pre-operative perfusion lung scans.

5. The apparatus according to claim 1, wherein the device is adapted to make use of the equation
EVLW=ITTV−ITBV|_{corrected},
wherein ITBV|_{corrected} is a value of the approximated intrathoracic blood volume (ITBVₐₚₚᵣₒₓ) corrected by the degree of lung resection (c).

6. The apparatus according to claim 2, wherein the device is adapted make use of equating the degree of lung resection (c) with a degree of reduction of pulmonary blood volume (PBV) caused by the lung resection.

7. The apparatus according to claim 6, wherein the apparatus is adapted to derive the global end-diastolic volume (GEDV) of the patient from the transpulmonary thermodilution curve and is adapted to make use of the equation
ITBV|_{corrected}=GEDV+PBV|_{corrected},
wherein PBV|_{corrected} is the value of the pulmonary blood volume (PBV) corrected by the degree of lung resection (c).

8. The apparatus according to claim 7, wherein the apparatus is adapted to make use of the equation
PBV|_{corrected}=((a−1)GEDV+b)(100%−c[%]),
wherein a and b are parameters.

9. The apparatus according to claim 8, wherein the parameters a and b are set to be
a=1.25 and b=0.0.

10. A computer program for assessing extravascular lung water volume (EVLW) of a patient after lung resection, comprising instructions adapted to carry out the following steps:
generating a transpulmonary thermodilution curve on basis of provided measurement data collected at the patient,
deriving an intrathoracic thermal volume (ITTV) of the patient and approximated intrathoracic blood volume (ITBVₐₚₚᵣₒₓ) from the transpulmonary thermodilution curve, and
determining the extravascular lung water volume (EVLW) by correcting by the degree of lung resection (c), wherein making use of the equation
EVLW=f(ITTV, ITBVₐₚₚᵣₒₓ, c), or
EVLW=f(ITTV, GEDV, c),
when run on a computer, GEDV being a global end-diastolic volume.

11. The computer program according to claim 10, further comprising instructions adapted to carry out the step of estimating the degree of lung resection (c) by making use of an empirical average value of the reduction of pulmonary perfusion of the resected lung compared to the pre-operative lung of the patient.

12. The computer program according to claim 10, further comprising instructions adapted to carry out the steps of:
in case of a resected right lung
estimating the degree of lung resection (c) as being 55%,
or in case of a resected left lung
estimating the degree of lung resection (c) as being 45%,
and/or in case of resected lobes
estimating the degree of lung resection (c) as being the sum of normal perfusion rates of the resected lobes.

13. The computer program according to claim 10, further comprising instructions adapted to carry out the step of estimating the degree of lung resection (c) by comparing the pulmonary perfusion before and after the lung resection, wherein the pulmonary perfusion values are provided by making use of pre-operative perfusion lung scans.

14. The computer program according to claim 11, further comprising having instructions adapted to carry out the step of making use of the equation
EVLW=ITTV−ITBV|_{corrected},
wherein ITBV|_{corrected} is the value of the approximated intrathoracic blood volume (ITBVₐₚₚᵣₒₓ) corrected by the degree of lung resection (c).

15. The computer program according to claim 11, further comprising instructions adapted to carry out the step of making use of equating the degree of lung resection (c) with the degree of reduction of pulmonary blood volume (PBV) caused by the lung resection.

16. The computer program according to claim 15, further comprising instructions adapted to carry out the steps of:
deriving the global end-diastolic volume (GEDV) of the patient from the transpulmonary thermodilution curve and
making use of the equation
ITBV|_{corrected}=GEDV+PBV|_{corrected},
wherein PBV|_{corrected} is the value of the pulmonary blood volume (PBV) corrected by the degree of lung resection (c).

17. The computer program according to claim 16, further comprising instructions adapted to carry out the step of making use of the equation
PBV|_{corrected}=((a−1)GEDV+b)(100%−c[%]),
wherein a and b are parameters.

18. The computer program according to claim 17, further comprising instructions adapted to carry out the step of setting the parameters a and b to be
a=1.25 and b=0.0.

19. A processor for implementing the computer program according to claim 10.

20. A method for assessing extravascular lung water volume (EVLW) of a patient after lung resection, comprising:
providing a transpulmonary thermodilution curve;
deriving a difference between the patient's intrathoracic thermal volume (ITTV) and approximated intrathoracic blood volume (ITBV) from the transpulmonary thermodilution curve, and
determining the extravascular lung water volume (EVLW) by correcting by the degree of lung resection (c), wherein making use of the equation
EVLW=f(ITTV, ITBVₐₚₚᵣₒₓ, c), or
EVLW=f(ITTV, GEDV, c).
